(19) United States
(12) Patent Application Publication
(10) Pub. No.: US 2023/0064524 A1
(43) Pub. Date: Mar. 2, 2023

(54) Synchronous Clinical Data Collection, Analysis and Reporting System and Method Therefor

(71) Applicants: Anna Barnacka, Cambridge, MA (US); Jal Mahendra Panchal, Somerville, MA (US)

(72) Inventors: Anna Barnacka, Cambridge, MA (US); Jal Mahendra Panchal, Somerville, MA (US)

(21) Appl. No.: 17/900,815

(22) Filed: Aug. 31, 2022

Related U.S. Application Data
(60) Provisional application No. 63/239,297, filed on Aug. 31, 2021.

(51) Int. Cl. G16H 40/63

(52) U.S. Cl. CPC G16H 40/63

(57) ABSTRACT

A synchronous clinical data collection, analysis and reporting system and method therefor are disclosed. The system collects and analyzes data from a patient, obtained via one or more medical devices. The medical devices include reference clinical devices installed at a medical setting that monitor vital signs and obtain signal data of the patient, and include one or more devices under test (DUT)s that obtain device-specific signal data. A clinical data recorder is configured to use signal data from one of the clinical devices as a reference signal to synchronize the signal data from each clinical device into clinical datasets. The reference signal is also used by the DUTs to synchronize the signal data from each of the DUTs into DUT datasets. Operators of the DUTs and an administrator of the system can access anonymized versions of the clinical datasets and/or the DUT datasets both during and after a medical session.

### RELATED APPLICATIONS

This application claims the benefit under 35 USC 119(e) of U.S. Provisional Application No. 63/239,297 filed on Aug. 31, 2021, which is incorporated herein by reference in its entirety.

### BACKGROUND OF THE INVENTION

Clinical trials and research of medical devices have well-defined protocols. These protocols keep the patient safe during testing, protect the data obtained during the trial in accordance with patient privacy and data security concerns, and separate the data obtained by each medical device under test (DUT) from data obtained by other medical devices used during the clinical trial. The collection of medical devices and other equipment used during a clinical trial or research session forms a clinical trial system.

A clinical research team of individuals operate as administrators of the clinical trial/research sessions. The administrators operate the medical devices, monitor the patient, and manage the recording and collection of the data obtained from the medical devices.

The clinical trials take place at medical facilities such as hospitals and clinics. Typically, a dedicated laboratory space or room within the medical facilities is established for each type of clinical trial. In one example, clinical trials of DUTs that are designed to monitor cardiovascular function of test patients often occur in cardiac catheter laboratories (“cath lab”).

During each clinical trial/research session, the administrators control the overall clinical research/trial and its operation, while on-site representatives for the DUTs control and manage the operation of the DUTs under the guidance of the administrators. These representatives are also known as operators of the DUTs.

The medical devices of the clinical trial systems (other than the DUTs) are also known as reference clinical devices. These reference clinical devices monitor vital signs of test patients and typically collect data from one or more bodily systems of the patient during the clinical trial or research session. These reference clinical devices are standardized and installed at the laboratory prior to introduction of the DUTs. Examples of reference clinical devices include an electrocardiogram system (ECG system) to monitor heart rate and respiration, one or more catheter systems to monitor blood pressure, and one or more X-ray systems.

A clinical patient monitoring and data recording system (“clinical data recorder”) is used to collect and record the data obtained by and sent from the reference clinical devices, while a separate recording system is typically used by the operators of the DUT(s) to collect and record the data obtained by and sent from the DUTs. These separate recording systems provide a boundary between the data obtained from the reference clinical devices and the data obtained from the DUT(s). Here, the data obtained is “raw” and does not identify the patient, which is important for accessibility and compliance with data privacy and security regulations. Examples of data privacy regulations include the General Data Protection Regulation 2016/679 (“GDPR”) adopted by the European Union, the California Consumer Privacy Act (CCPA) of 2018 in California in the United States, and the nationwide Health Insurance Portability and Accountability Act (HIPAA) introduced in the United States in 1996.

The clinical data recorder has interfaces for each reference clinical device and monitors and records time-stamped analog output data from each reference clinical device. The clinical data recorder also typically has a defibrillator interface that provides a defib sync port signal that is normally used as a timing reference for an external defibrillator The external defibrillator can restore normal operation of the test patient's heart in the unlikely event that the patient's heart stops beating or beats erratically during the clinical trial.

One example of a clinical data recorder is a General Electric (GE) Transport Remote Acquisition Monitor (TRAM). The GE TRAM is a medical grade computer system with a chassis-based design that accepts one or more modular computer processing boards. These modular computer processing boards insert into slots of the chassis and are also known as modules. The chassis also has a backplane that allows sharing of power, control signals and some data between the modules.

Each module in the GE TRAM has a specific purpose. Most of the modules are feature-specific modules that are designed to interface with and support collection and recording of data from a specific reference clinical device, while other modules provide capabilities that span across multiple modules. Examples of feature-specific modules include multi-lead/multi-wire ECG, invasive blood pressure (BP) measurement using catheters, pulse oximetry (“SpO2”), impedance for respiration, and non-invasive blood pressure (“NIBP”) modules. The GE TRAM also has a defibrillator module with a configurable defib sync port.

The clinical data recorder records the time-stamped analog output data from each reference clinical device, and includes the time-stamped analog output data from each reference clinical device into an output dataset known as a clinical dataset.

The clinical data recorder is also designed to interface with an electronic records management system. The records management system is either installed at the medical facility where the clinical trial occurs or is accessible externally via a secure network connection. The records management system stores the time-stamped clinical datasets sent from the clinical data recorder and maintains security and privacy over the data. One example of a records management system used at many US hospitals and clinics is Epic EHR.

The separate recording system used by the operators of the DUT(s) to collect and record the data obtained by and sent from the DUTs is also known as a DUT recorder. A separate DUT recorder is used for each DUT device.

Each DUT recorder has interfaces to collect and record the data obtained by and sent from each DUT, also known as DUT datasets. However, the DUT recorder cannot access the medical facility's records management system. As a result, the DUT datasets and the clinical dataset are kept separate.

### SUMMARY OF THE INVENTION

The existing clinical trial/research systems have limitations. In general, each of the reference clinical devices and the DUTs have different levels of response and delay when obtaining their signals, and obtain their signals using a separate internal reference clock. As a result, the signals within the clinical dataset typically have different timestamps and are not synchronized with respect to one another, and the signals within the DUT datasets typically have different timestamps and are not synchronized with respect to one another. Correspondingly, the signals across the clinical dataset and the DUT datasets are thus also not synchronized.

The lack of signal synchronization within the clinical datasets and within each of the DUT datasets, and the lack of signal synchronization across the clinical dataset and the DUT datasets, can affect the usability of the data collected and the results/conclusions reached upon analyzing the data. The administrators of the existing clinical trial/research systems collect the datasets after the research/trial has completed, analyze the datasets, and provide results in response to the analysis. However, the analysis and results are often significantly impacted by the lack of signal synchronization/differences in timestamps among the signals, especially as the time differences between the signals increases. This lack of signal synchronization/level of mis-synchronization also cannot be cured after data collection. As a result, the usability of the data collected (and thus the accuracy of results/conclusions reached) can be reduced, which challenges the ability of the DUT manufacturers to develop accurate algorithms and solutions.

Another limitation is that during the research/trial, the operators of the DUTs usually have access only to the live catheter data from the catheter system reference clinical device. The operators of the DUTs not only do not have access to other clinical device data during the trial (such as the ECG signals from the ECG system), the operators also cannot access the recorded clinical datasets created or otherwise produced by the clinical data recorder after the research/trial has concluded. As a result, the operators of the DUTs and other stakeholders must wait weeks or months for the administrators to analyze and interpret the DUT datasets in light of the clinical dataset in order to make any conclusions concerning the efficacy of the DUT(s).

Moreover, as discussed previously, the lack of signal synchronization/level of mis-synchronization within the clinical dataset and the DUT datasets themselves can affect the analysis performed upon the datasets and the results reached by the administrators. For example, experimentation has shown that time differences between signals from different clinical devices within the clinical dataset can be on the order of seconds. At the same time, human physiology can change significantly between heartbeats. While the heartbeats are on the order of seconds, there are some cardiac events or measurements that occur between or across heartbeats that are on the order of tens milliseconds (mSec). In one example, the variability between heartbeats, also known as heart rate variability (HRV), is typically on the order of tens of mSec, while resting BP has been shown to change as much as 20 mmHG within a period of less than three seconds. Poorly synchronized datasets could cause the researchers to either not detect these events, or fail to detect correlations in multiple signals when these events occur.

Still another limitation is that the results of the clinical trials are typically limited to a small set of plotted images of the DUT datasets and the clinical dataset that the administrators of the clinical research/trial deem as relevant. Such a limited set of images provides an imperfect and incomplete assessment of the capabilities and efficacy of the DUTs.

It is therefore an object of the present invention to provide a clinical data collection, analysis and reporting system (“clinical data system”) that synchronizes the DUT datasets with the clinical datasets, in real-time, during the clinical trial or research session. The proposed clinical data system also allows remote personnel such as other researchers and DUT stakeholders to access the clinical datasets and the DUT datasets in real-time and communicate with personnel in real-time during the clinical research/trial. Here, the remote personnel can assess the quality of the datasets, and based upon the clinical datasets and/or the DUT datasets, provide interactive feedback and instruction to the on-site administrator of the clinical data system and the operators of the DUTs concerning valid use and operation of the DUTs and the reference clinical devices to achieve best quality or acceptable data.

For this purpose, in one embodiment, the proposed clinical data system employs a defibrillator synchronization output port (“defib sync port”) signal of the clinical data recorder to use as a reference signal. This reference signal is used to synchronize the signals sent from each DUT and thus synchronize the DUT dataset for each DUT. The same reference signal is also used to synchronize the signals in the clinical dataset. Preferably, the reference signal is/are ECG signals from an ECG system reference clinical device. For this purpose, the clinical data recorder is configured such that its defib sync port provides the ECG signals as a reference signal.

The clinical data recorder also includes the defib sync port reference signal in the clinical dataset. In this way, the same reference signal within the clinical dataset can be used to synchronize the signal data in each DUT dataset collected over the same time period as each clinical dataset, in real-time, during the clinical trial.

The proposed clinical data system also provides the operators of the DUT(s) with access to the clinical dataset both during and after the clinical research/trial. In this way, the operators of the DUT(s) can analyze their DUT datasets with the clinical dataset independently from the administrators and draw their own conclusions regarding the efficacy of the DUTs. Moreover, the analysis and conclusions can take place over possibly days or hours rather than months, as in existing research or clinical trial/study systems. Additionally, the remote personnel can also access the clinical dataset and the DUT dataset(s).

At the same time, the proposed clinical data system maintains the data security and privacy of the clinical datasets as in the existing clinical trial systems. This is because the clinical datasets include data that carries no identifiable information that directly ties the data to individuals. Rather, the data is anonymized/tagged only with unique alphanumeric strings. Only the records management system includes the mappings between the alphanumeric strings in the data and the identities of the test patients. Though the proposed clinical data system allows operators of the DUTs to access the clinical datasets during the clinical trial, the clinical data system cannot access the records management system of the clinical setting. As a result, data security and patient privacy is maintained.

The proposed clinical data system has many advantages over the existing clinical trial systems. In one example, the DUT dataset from each DUT is/are synchronized with a reference signal of the clinical datasets, in real-time. Another example is that the operators of the DUTs have access to all data collected during the clinical trial. Not only can the researchers make more informed conclusions regarding the efficacy of their DUTs, but the analyses performed and any conclusions reached can be performed independently from the administrators of the research/trials and be completed in possibly weeks or even days. In yet another example, the clinical datasets and the DUT datasets can be scrubbed to eliminate even the alphanumeric identifiers. This allows the data to be shared for peer review and independent confirmation without breaching patient privacy and data security protocols. In still other examples, the reference signal can be used to calibrate the other clinical devices used during the clinical trial, and to also calibrate each DUT.

In addition to clinical trial/research sessions, the proposed clinical data system can also be used to collect, synchronize, calibrate and record signal data from reference clinical devices and from DUT devices during surgical and other medical procedures. For this purpose, the broader term “medical setting” refers to any of the following: clinical trial setting, research setting, surgical/hospital setting, laboratory setting, outpatient setting or other setting in which the proposed clinical data system can operate. The duration of time that the proposed clinical data system operates at a medical setting is known as a medical session.

In general, according to one aspect, the invention features a synchronous clinical data collection, analysis, and reporting system (“clinical data system”). The clinical data system includes a clinical data recorder, one or more devices under test DUT and a computing unit. The clinical data recorder collects and records signal data of a patient obtained by and sent from reference clinical devices during a medical session, synchronizes the signal data from each of the reference clinical devices using a reference signal, and creates clinical datasets that include the signal data from each of the reference clinical devices and the reference signal. The one or more DUTs each obtain signal data from the patient during the session, where each DUT synchronizes its signal data using the reference signal and creates DUT datasets that each include the signal data from each DUT and the reference signal. The computing unit receives the clinical datasets from the clinical data recorder and the DUT datasets sent from each of the DUTs, computes a normalized reference signal for each DUT dataset, and synchronizes the signal data of each DUT dataset with the signal data of the clinical datasets using the normalized reference signal computed for each DUT dataset.

In one embodiment, the clinical data recorder is configured to obtain the reference signal from the signal data of one of the reference clinical devices. In one example, the reference signal from the signal data of one of the reference clinical devices is/are ECG signals of an ECG system reference clinical device.

In another embodiment, the clinical data recorder is configured to use the reference signal from the signal data of one of the DUTs. In one example, the reference signal from the signal data of one of the DUTs is/are earbud signals detected by and sent from an earbud system DUT.

Typically, the computing unit records the DUT datasets and provides an interface that enables operators of the DUTs to access anonymized versions of the DUT datasets in real time during the session and after all DUT datasets have been received. Additionally and/or alternatively, the computing unit provides an interface that enables an administrator of the system to access anonymized versions of the clinical datasets in real time during the session and after all clinical datasets have been received.

Preferably, the computing unit computes a normalized reference signal for each DUT dataset by identifying DUT datasets and one or more clinical datasets obtained over substantially a same time period, and calculating and subtracting delays between the reference signal of the clinical datasets and the reference signal of each DUT dataset obtained over substantially the same time period, the result of which is the normalized reference signal for each DUT dataset.

Additionally, the computing unit might calibrate the signal data of each DUT dataset upon determining that delays between the signal data and reference signal of each DUT dataset collected over a same time period are above a threshold value. In one example, when the reference signal in each DUT dataset is an ECG signal from an ECG system, the computing unit either calibrates each DUT dataset upon determining that delays between the signal data and reference signal of each DUT dataset collected over a same time period are above a threshold value, or upon determining that delays between the signal data and the reference signal of each DUT dataset collected over the same time period change more than a threshold amount over a range of successive heartbeats in the ECG signal as the reference signal.

In another example, the computing unit calibrates each DUT dataset by either identifying a signal feature in or introducing a signal feature into both the signal data and the reference signal of each DUT dataset collected over the same time period, and then identifying a difference in time when the signal feature occurs in the signal data and in the reference signal.

Additionally, the computing unit might calibrate each clinical dataset upon determining that delays between the signal data and reference signal of each clinical dataset are above a threshold value. In one example, when the reference signal in each clinical dataset is an ECG signal from an ECG system, the computing unit either calibrates the clinical dataset upon determining that delays between the signal data and reference signal of each clinical dataset are above a threshold value, or upon determining that delays between the signal data and the reference signal of each clinical dataset change more than a threshold amount over a range of successive heartbeats in the ECG signal as the reference signal.

In another example, the computing unit calibrates each clinical dataset by either identifying a signal feature in or introducing a signal feature into both the signal data and the reference signal of each clinical dataset, and then identifying a difference in time when the signal feature occurs in the signal data and in the reference signal of each clinical dataset.

In general, according to another aspect, the invention features a synchronous clinical data collection, analysis, and reporting method. The method comprises collecting signal data of a patient obtained by and sent from reference clinical devices during a medical session, synchronizing the signal data from each of the reference clinical devices using a reference signal, and creating clinical datasets that include the signal data from each of the reference clinical devices and the reference signal. The method also comprises collecting signal data of the patient obtained by and sent from one or more devices under test (DUT) during the session, synchronizing the signal data from each DUT using the same reference signal, and creating DUT datasets for each DUT that include the signal data from each DUT and the reference signal. The method also comprises computing a normalized reference signal for each DUT dataset, based on differences in time between the reference signals of each DUT dataset collected over a same time period and the reference signals of clinical datasets collected over the same time period as each DUT dataset. The method also comprises synchronizing the signal data of each DUT dataset with the signal data of the clinical datasets using the normalized reference signal computed for each DUT dataset.

The method further comprises recording the DUT datasets and providing an interface that enables operators of the DUTs to access anonymized versions of the DUT datasets in real time and after all DUT datasets have been recorded. The method also further comprising recording the clinical datasets and providing an interface that enables an administrator to access anonymized versions of the clinical datasets in real time and after all clinical datasets have been recorded.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic diagram of an exemplary clinical trial data collection and reporting system (“clinical data system”) constructed in accordance with the present invention;

FIG. 2A shows a method of operation of the clinical data system that synchronizes signal data from the clinical devices and the DUTs using a common reference signal, for signal data collected over a time window of 15 seconds;

FIG. 2B shows a calibration method of the clinical data system that operates over the same time window as in FIG. 2A; and

FIG. 3 is an exemplary device under test (DUT) dataset for an earbud system DUT in FIG. 1, where the DUT dataset was created in accordance with the method of FIG. 2A and is shown over the same 15 second time window.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

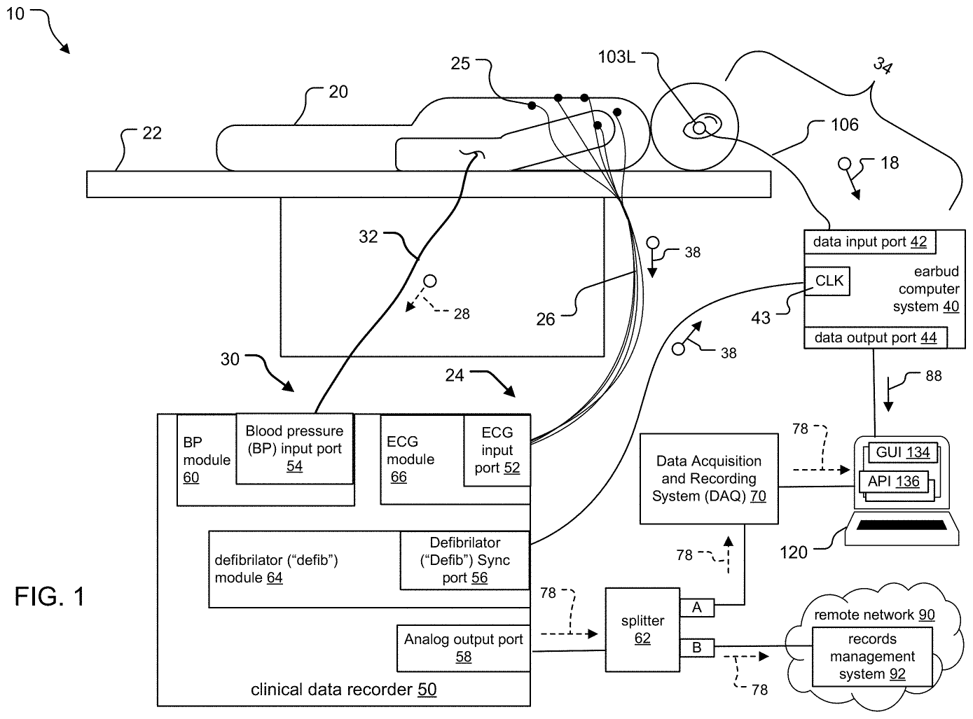

FIG. 1 shows an exemplary clinical data system 10. The system 10 monitors, records and stores medical data of a test patient 20. The test patient 20 is in a medical setting and is the participant of a medical session for one or more medical/wearable devices under test (DUTs). The medical setting is established to study, test, and/or certify one or more DUTs in a controlled environment.

The clinical data system 10 has various components. These components include a clinical data recording system (“clinical data recorder”) 50, one or more reference clinical devices, one or more DUTs, a data acquisition and recording system (DAQ) 70, a computing unit 120 and a remote network 90. The remote network 90 includes a records management system 92. In one example, as shown, the computing unit 120 is a laptop computer.

The clinical data recorder 50 includes a BP module 60, an ECG module 66, a defibrillator (“defib”) module 64 and a multichannel analog output port 58. The BP module 60 has a BP input port 54, the ECG module 66 has an ECG input port 52 and the defib module 64 has a defibrillator (“defib”) sync port 56. In the illustrated example, the clinical data recorder 50 is a GE TRAM.

In the illustrated example, the test patient 20 is at rest upon a hospital bed 22. The reference clinical devices and a DUT are attached to the patient 20. The reference clinical devices are an ECG system 24 and a catheter system 30. The DUT is an earbud system 34.

More detail for the reference clinical devices is as follows. The ECG system 24 includes electrodes 25, wires 26 and a monitor device (here, the ECG module 66 of the clinical data recorder 50). The electrodes 25 attach to the patient's skin and the wires 26 connect to each of the electrodes 25 and to the ECG port 52 of the ECG module 66. The electrodes 25 are each attached to different locations on the patient's skin that are near the heart and major arteries of the patient 20. The electrodes 25 detect electrical signals associated with cardiovascular activity, also known as ECG signals 38. The ECG signals 38 are sent via the wires 26 to the ECG module 66 as the monitor device of the ECG system 24 for filtering, amplification and recording.

The catheter system 30 includes a catheter 32 and a monitor device (here, the BP module 60 of the clinical data recorder 50). The catheter 32 is inserted into a major vein or artery of the patient's arm and guided until the end of the catheter 32 rests within a portion of the circulatory system to measure its blood pressure and flow. In the example, the catheter 32 rests within the aorta of the patient's heart to measure a central aortic pressure. Alternatively, the end of the catheter 32 might be placed in the patient's left ventricle to measure a left ventricular pressure. These pressure signals are also known as blood pressure signals, or BP signals 28. The BP signals 28 are sent via the catheter 32 to the BP module 60 as the monitor device of the catheter system 30 for recording.

The earbud system 34 includes earbuds 103 and an earbud computer system 40 with various ports. Only a left earbud 103L is shown; typically, both a right earbud and the left earbud 103L are included. The ports of the earbud computer system 40 include a data input port 42, a clock input port (“CLK port”) 43and a data output port 44. The earbuds 103 are inserted into or are placed at ear canals of the patient 20, and the earbud connection 106 connects to the data input port 42 of the earbud computer system 40.

Each of the earbuds 103 include sensors that detect sounds including infrasounds. Infrasounds are low-frequency sounds that are below 20 Hz. The infrasounds are generated from and associated with various bodily functions of the patient 20. These bodily functions include cardiovascular operation such as myocardial contraction, blood flow, arterial wall vibrations, and skeletal muscle motion, in examples. The earbuds 103 detect the sounds including infrasounds from the body of the patient 20 in the ear canals, and send an electronic representation of the detected sounds, also known as earbud signals 18, over the earbud connection 106 to the earbud computer system 40.

The clinical data recorder 50 monitors and records signals sent from the reference clinical devices. The defib sync port 56, as its name suggests, is configured by default to provide a reference signal to an external defibrillator.The external defibrillator can restore normal operation of the test patient's heart in the unlikely event that the patient's heart stops beating or beats erratically during the clinical trial. The GE TRAM, for example, has a module with a configurable defib sync port.

The clinical data recorder 50 is also configured such that the ECG signals 38 received at the ECG module 66 are internally mapped to the defib sync port 56. In this way, the signals outputted from the defib sync port 56 for synchronizing data from the DUTs are the ECG signals 38.

The computing unit 120 includes a graphical user interface (“GUI”) 134 and one or more application programming interfaces (“API”) 136. The GUI 134 and the APIs 136 can be software and/or firmware loaded into a memory of the computing unit 120. The GUI 134 and the APIs 136 execute upon a processor of the computing unit 120. Typically, an operating system of the computing unit 120 loads instructions of the GUI 134 and the APIs into the memory, and the operating system instructs the processor to execute the instructions.

Operators of the DUT devices and the administrator of the clinical data system 10 that are authorized to access the computing unit 120 can do so via the GUI 134. Based upon pre-established access control lists and protocols, the administrator of the system 10 can access the clinical devices and configure the system 10 via the GUI 134 and/or the APIs 136, and the operators of the DUTs can access their respective DUT devices and configure the DUTs via the GUI 134 and/or the APIs 136.

The administrator of the system 10 and the operators of the DUTs may additionally be authorized to access information obtained and recorded during a medical session, and after the session has concluded, via the one or more APIs 136. In one implementation, the computing unit 120 presents a separate API 136 for the administrator to access anonymized versions of clinical datasets obtained and/or recorded from each clinical device, and also presents separate APIs for the operators of each DUT device to access anonymized versions of DUT datasets obtained and/or recorded from each respective DUT device.

In another implementation, the operators of at least some of the DUTs may also be granted access to the anonymized versions of the clinical datasets. For this purpose, the administrator of the system 10 would typically configure the computing unit 120 via the GUI 134 and the APIs 136 to provide the operator(s) with anonymized access to the clinical datasets in accordance with agreed-upon access controls and data security and privacy obj ectives.

The components of the clinical data system 10 are arranged as follows. The BP input port 54 and the ECG input port 52 of the clinical data recorder 50 respectively connect to the catheter 32 and the wires 26 of the ECG system 24. These connections enable the catheter 32 to send the BP signals 28 and the electrodes 25 to send the ECG signals 38 via the wires 26 to the clinical data recorder 50.

The earbud computer system 40 connects to the clinical data recorder 50 and the computing unit 120. The CLK port 43 connects to the defib sync port 56 of the clinical data recorder 50. Here, the clinical data recorder 50 is configured such that a copy of the ECG signals 38 are sent to the defib sync port 56. The CLK port 43 then receives the ECG signals 38 from the defib sync port 56. The data output port 44 connects to the computing unit 120.

The clinical data recorder 50 also connects to the records management system 92 and the DAQ 70. In more detail, the analog output port 58 of the recorder 50 connects to a splitter 62. The splitter typically has two ports, A and B. Port A connects to the DAQ 70, while port B connects to the records management system 92.

The DAQ 70 then connects to the computing unit 120. In one example, the connection is a high-speed serial connection, such as a USB 3.0 or 3.1 connection.

The exemplary clinical data system 10 correlates the earbud signals 18 from the earbud system 34 DUT with myocardial mechanics of the patient 20 undergoing cardiac catheterization. The myocardial mechanics, in one example, are expressed as the ECG signals 38 from the ECG system 24 and the BP signals 28 from the catheter system 30.

The clinical data system 10 generally operates as follows. The earbud computer system 40 continuously receives the earbud signals 18 sent from the earbuds 103 at the data input port 42. The earbud computer system 40, via signals at its CLK port 43, synchronizes the received earbud signals 18. Here, the signals received at the CLK port 43 are the ECG signals 38 sent from the defib sync port 56 of the clinical data recorder 50. The earbud computer system 40 then pairs the ECG signals 38 with the now synchronized earbud signals 18 to create a DUT dataset 88. The earbud computer system 40 then sends the DUT dataset 88 via the data output port 44 to the computing unit 120 for recording, analysis and storage.

At the clinical data recorder 50, the ECG signals 38 and the BP signals 28 are received at the ECG input port 52 and BP input port 54, respectively. The clinical data recorder 50 includes these signals in a clinical dataset 78 and forwards the clinical dataset 78 to its multichannel analog output port 58. The splitter 62 sends the clinical dataset 78 to both the DAQ 70 and to the records management system 92. The DAQ 70 digitizes the clinical dataset 78 and sends the digitized version to the computing unit 120 for recording, analysis and storage. In this way, the entirety of the clinical datasets 78 and the DUT datasets 88 recorded during the research/trial are available to the operators of the DUT(s) at the computing unit 120 for analysis.

Additionally and/or alternatively, the splitter 52 can have a third port (not shown) that provides the clinical dataset 78 as output. In one implementation, the third port can connect to the CLK port 43 of the earbud computer system 40 instead of connecting to the defib sync port 56 and receiving the ECG signal 38. The earbud computer system 40 (or any other DUT) can then be configured to receive its synchronization signal from the clinical dataset 78 (typically, from the reference signal of the clinical dataset).

While only a single DUT (here, the earbud system 34 DUT) is shown, it can be appreciated that the clinical data system 10 can support multiple DUTs during the same medical session. When additional DUTs are included in the clinical data system 10, operators of each additional DUT connect the clock/synchronization input port of the DUT to the defib sync port 56. The ECG signals 38 that the clinical data recorder 50 sends over the defib sync port 56 are then used by each DUT to synchronize its DUT-specific data. The operators of the additional DUTs also connect the output ports of the additional DUTs to the computing unit 120.

The clinical data system 10 can also analyze data from different medical devices (e.g., the signal data of clinical devices and the signal data of DUTs) in real-time and extract, derive, or otherwise calculate various metrics from the analysis. These metrics can be device-specific, or more importantly, can be cross-device metrics that require information from multiple devices of different types. The clinical data system 10 can provide results of the analysis such as the cross-device metrics to the administrator of the system and/or the operators of the DUTs both during and after completion of a medical session.

In examples, the calculation of systolic and diastolic time duration cross-device metrics, also known as systole and diastole, respectively, typically require ECG signals 38 from an ECG system 24 reference clinical device and additional signal data from a DUT device. The additional signal data and DUT device might be echo signals from an echocardiogram system, BP signals 28 from a catheter system 30, earbud signals 18 from an earbud system 34, or possibly even combinations of the signal data from two or more of these DUT systems.

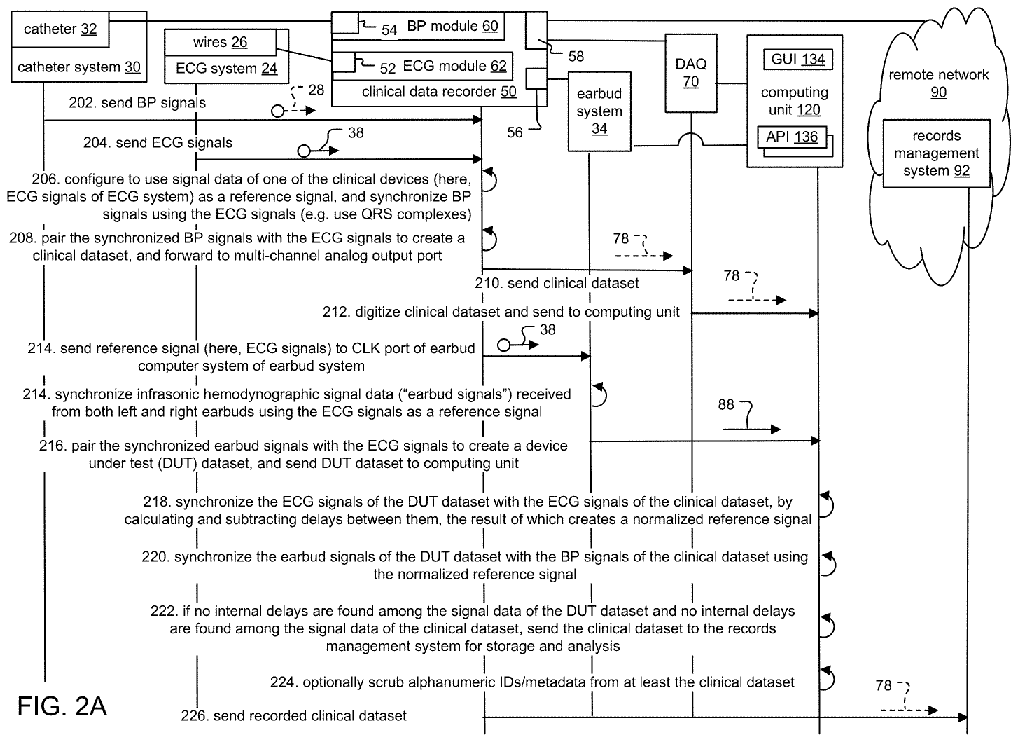

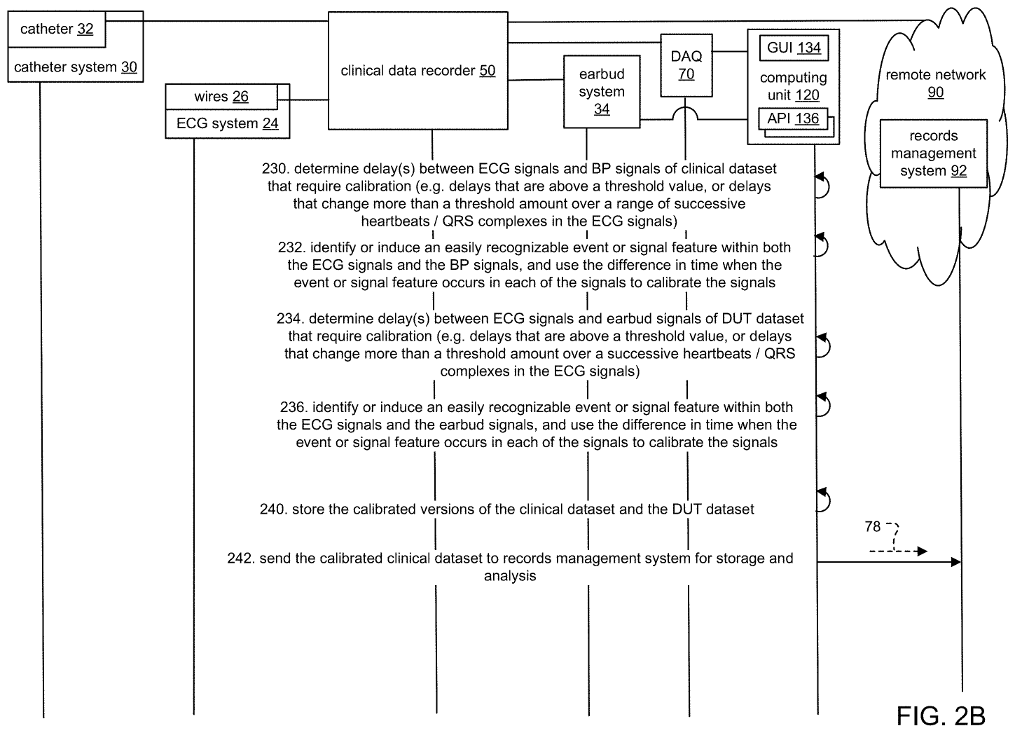

As with the earbud system 34 DUT, each additional DUT synchronizes its DUT-specific signals using the ECG signals 38, and includes the ECG signals 38 as the reference signal with the now synchronized DUT-specific signals 18 to form a DUTdataset specific to each DUT. The computing unit 120 then records and analyzes the DUT-specific datasets from each of the additional DUTs, as well as the DUT dataset 88 from the earbud system 34 DUT. More detail for operation of the clinical data system 10 is provided in the methods of FIG. 2A and 2B, the descriptions of which are included herein below.

FIG. 2A shows a method of operation of the clinical data system 10.With reference to FIG. 1, the method illustrates how the system 10 synchronizes signal data from the ECG system 24 and catheter system 30 reference clinical devices, using a reference signal based upon the signal data of one of the clinical devices (here, using the ECG signals 38 of the ECG system 24). The method also shows how the same reference signal is used to synchronize the signal data from the exemplary earbud system 34 DUT device.

Because medical sessions can last for a period of time as short as few minutes, for a period of hours, or for long as two or more days, it can be appreciated that the system 10 collects, synchronizes, and records many clinical datasets 78 and DUT datasets 88 during a medical session. For this purpose, the system 10 typically selects a repeating window of time, such as 15 seconds or as much as 30 seconds, to collect and synchronize the signal data from the clinical devices and to create a clinical dataset 78 that includes the signal data and the reference signal used to synchronize the signal data. The system 10 also collects and synchronizes the signal data from each of the DUTs over the same time window, and creates the DUT datasets for each DUT for the same time window.

In the illustrated example, the operation of the clinical data system 10 is shown over a selected time window of 15 seconds. During this time period, the system 10 collects and synchronizes the signal data from the clinical devices and includes the signal data into a single clinical dataset 78. In a similar vein, over the same time period, the system 10 collects and synchronizes the signal data from each of the DUTs and includes the signal data into a separate DUT dataset 88 for each DUT. The method starts at step 202.

At step 202, the catheter system 30, via the catheter 32, sends BP signals 28 of the patient 20 to the BP input port 54 of the clinical data recorder 50. The BP signals 28 are the signal data obtained by and sent from the catheter system 32. Here, the BP input port 54 is a component of the BP module 60 that inserts into the clinical data recorder 50. While only one catheter 32 is shown, it is often the case that two or possibly more catheters might be used.

When two catheters 32 are used, one catheter is threaded through the patient's circulatory system and into the aorta to measure a central aortic pressure, while the second is threaded into the left ventricle to measure a left ventricular pressure. Typically, each catheter interfaces with a BP input port 54 of a separate BP module 60 of the recorder 50.

According to step 204, the ECG system 24 sends its ECG signals 38 via the wires 26 to the ECG input port 52 of the clinical data recorder 50. The ECG signals 38 are the signal data obtained by and sent from the ECG system 24. The ECG signals 38 are used to monitor the patient's cardiovascular system during the clinical research/trial, and can also be used as a reference signal or “clock signal” for synchronizing other signals and devices in the clinical data system 10.

By way of background, most medical devices do not set their clocks using an external time reference, such as a network time reference and there is no widely adopted standard for medical device time management. Typically, the internal clocks of the medical devices are set manually twice a year for daylight savings time, or when the medical devices are serviced. The lack of automatic clock-setting capabilities in most medical devices, and the lack of time synchronization among the different medical devices used in a typical medical setting can result in inaccurate timestamps of clinical data obtained during a clinical research/trial and recorded to the electronic record management system 92. These inaccuracies also complicate the synchronization of signals within datasets, such as within the clinical datasets 78 and the DUT datasets 88 when analyzing the datasets and events that occur within the datasets 78, 88.

The ECG signals 38 include a periodic QRS complex that makes the ECG signals 38 especially suited for use as a reference clock for synchronization purposes. The QRS complex is the salient signal feature of an ECG signal 38 and represents depolarization of the ventricles during each heart cycle. The QRS duration thus represents the time over which ventricular depolarization occurs. Because of its prominence within the ECG signal 38 and its occurrence within each heart cycle, the QRS complex (and thus the ECG signal 38 itself) is a reliable clock signal.

In step 206, the clinical data recorder 50 configures the defib module 64 to use the signal data from one of the clinical devices (here, the ECG signals 38 of the ECG system 24) as a reference signal or clock at the defib sync port 56. The clinical data recorder 50 then synchronizes the BP signals 28 with the ECG signals 38 (e.g., using the QRS complexes of the ECG signals 38).

To configure the ECG signals 38 to use as a reference signal, in one example, the clinical data recorder 50 receives a command from the administrator of the clinical data system 10 at a user interface of the clinical data recorder 50, such as via the GUI 134. In response to receiving the command, a controller or other processor of the clinical data recorder 50 executes an instruction in memory of the recorder 50 that directs the defibrillator module 64 of the clinical data recorder 50 to “map” its input to the ECG signals 38. As a result, a copy of the ECG signals 38 are sent to the defib sync port 56.

In another embodiment, DUTs other than (or in addition to) the earbud system 34 can be tested. Here, the DUTs might include photoplethysmography (PPG) based wearable devices, ECG systems 24, catheter systems 30, magnetic resonance imaging systems (MRI), x-ray systems, EEG systems, or possibly any other type of medical device. The DUTs each use the signal at the defib sync port 56 as a reference signal for synchronizing the output signals provided by each DUT.

In still another embodiment, an earbud system 34 installed at the site of the clinical research/trial operates as a clinical device, and can be used to provide a reference signal for synchronizing the other clinical devices and/or the one or more DUTs. Here, the DUTs can be any type of medical device and the clinical data recorder 50 would be configured to use the earbud signals 18 of the earbud system 34 as a reference signal.

According to step 208, the clinical data recorder 50 pairs the synchronized BP signals 28 with the ECG signals 38 to create a clinical dataset 78, and forwards the dataset 78 to the multi-channel analog output port 58. In more detail, the clinical dataset 78 includes the signal data from both the ECG system 24 and the catheter system 30 (i.e., the BP signals 28 and the ECG signals 38, respectively) and also includes the reference signal that the clinical data recorder 50 used to synchronize the signal data (here, the ECG signals 38). The clinical data recorder 50 then sends the clinical dataset 78 to the DAQ 70 in step 210. Here, a copy of clinical dataset 78 is sent from port A of the splitter 62 to the DAQ 70. The DAQ 70, in step 212, digitizes the clinical dataset 78 and sends it to the computing unit 120 for analysis and storage.

According to step 214, the earbud system 34 as a DUT synchronizes the infrasonic hemodynographic earbud signals 18 received from both left and right earbuds 103 with the same reference signal. For this purpose, the earbud system 34 receives the ECG signals 38 over the connection between the defib sync port 56 and the CLK port 43 to use as a reference signal, synchronizes the earbud signals 18 using the reference signal. According to step 216, the earbud system 34 pairs the (now synchronized) earbud signals 18 with the ECG signals 38 reference signal to create a DUT dataset 88 and sends the DUT dataset 88 to the computing unit 120.

In step 218, the computing unit 120 synchronizes the ECG signals 38 of the DUT dataset 78 with the ECG signals 38 of the clinical dataset 78. For this purpose, the computing unit can calculate and subtract delays between them. Such an operation eliminates clock skew between the ECG signals 38 across all of the datasets 78, 88. The result of this operation creates a normalized reference signal.

According to step 220, the computing unit 120 synchronizes the earbud signals 18 of the DUT dataset 88 with the BP signals 28 of the clinical dataset 78 using the normalized reference signal calculated in step 218.

Then, if the computing unit 120 in step 222 determines that no internal delays are found among the signals of the DUT dataset 88 and that no internal delays are found among the signals of the clinical dataset 78, the computing unit 120 stores the clinical dataset 78 and the DUT datasets 88 for subsequent analysis. In step 224, the computing unit 120, via the APIs 136, might scrub alphanumeric IDs/metadata from at least the clinical datasets 78 and possibly also from the DUT datasets 88 to create anonymized versions of the datasets 78, 88.

In step 226, the clinical data recorder 50 sends the recorded clinical dataset 78 to the records management system 92 for analysis and storage.

FIG. 2B shows a calibration method for the clinical data system 10. Here, the computing unit 120 uses the same reference signal used in the method of FIG. 2A to now calibrate the signal data of the clinical datasets 78, and to also calibrate the signal data of each DUT dataset 88.

Calibration of the signal data of each clinical dataset 78 is typically required when delays are found among the signal data that exceed a threshold amount. In more detail, for each clinical dataset 78, the computing unit 120 compares each instance of signal data (each of which was obtained from different reference clinical devices) against one another, and determines whether any of the differences are above a threshold value.

The threshold value selected is typically application-dependent. In cardiology applications, for example, experimentation has shown that when the reference signal used to synchronize each clinical dataset 78 is/are the ECG signals 38 from the ECG system 24, or is/are the earbud signals 18 from the earbud system 34, the computing unit 120 typically uses a threshold value in a range between 1 mSec and 10 mSec, inclusive. This is because there are several features in the signal data that are in a range between 10 mSec and 150 mSec. This in contrast to traditional clinical trial/research systems, which provide a coarser level of synchronization of the signal data that results in delays within the signal data that are typically in a range between tens of milliseconds to as much as three or four seconds, in examples.

In a similar vein, calibration of the signal data of each DUT dataset 88 is typically required when delays are found among the signal data that also exceed the same threshold amount, or exceed a different threshold amount than that used for determining whether calibration of the signal data of the clinical dataset 78 is required. While only one DUT dataset 88 is shown, calibration in general accesses multiple DUT datasets collected over the same time window, and compares the signal data from each of the DUT datasets 88 against one another to determine determine whether any of the differences are above a threshold value. As with the method of FIG. 2A, the method of FIG. 2B operates over the same time window of 15 seconds. The method begins at step 230.

In step 230, the computing unit 120 determines delay(s) between the reference signal (here, the ECG signals 38) and the BP signals 28 of the clinical dataset 78 that require calibration. In examples, the delays that are indicative of mis-calibration/the need to calibrate are either above a threshold value, or are indicated by changes in time that are more than a threshold amount over a range of successive heartbeats/QRS complexes in the reference ECG signals 38. In examples, the threshold value can be as small as one mSec, in a range between 1 mSec and 10 mSec, on the order of tens of mSec, or possibly hundreds of mSec. In another example, instead of using ECG signals 38, earbud signals 18 from the earbud system 34 might be used for calibration purposes.

In step 232, the computing unit 120 identifies or induces an easily recognizable event or signal feature within both the ECG signals 38 and the BP signals 28 of the clinical dataset 78, and uses the difference in time when the event or signal feature occurs in each of the signals to calibrate the signals 28, 38. For this purpose, in examples, the computing unit 120 might introduce a reference tone at a fixed frequency or a chirped signal into the signals 28, 38 of the clinical dataset 78.

In a similar vein, according to step 234, the computing unit 120 determines delay(s) between the ECG signals 38 and the earbud signals 18 of the DUT dataset 88 that require calibration (e.g., delays that are above a threshold value, or delays that change more than a threshold amount over a range of successive heartbeats/QRS complexes in the ECG signals). In step 236, the computing unit 120 identifies or induces an easily recognizable event or signal feature within both the ECG signals 38 and the earbud signals 18, and uses the difference in time when the event or signal feature occurs in each of the signals to calibrate the signals 38, 18.

The computing unit 120 then stores the calibrated versions of the clinical dataset 78 and the DUT dataset 88 in step 240, and sends the calibrated clinical dataset to the records management system 92 for storage and analysis in step 242.

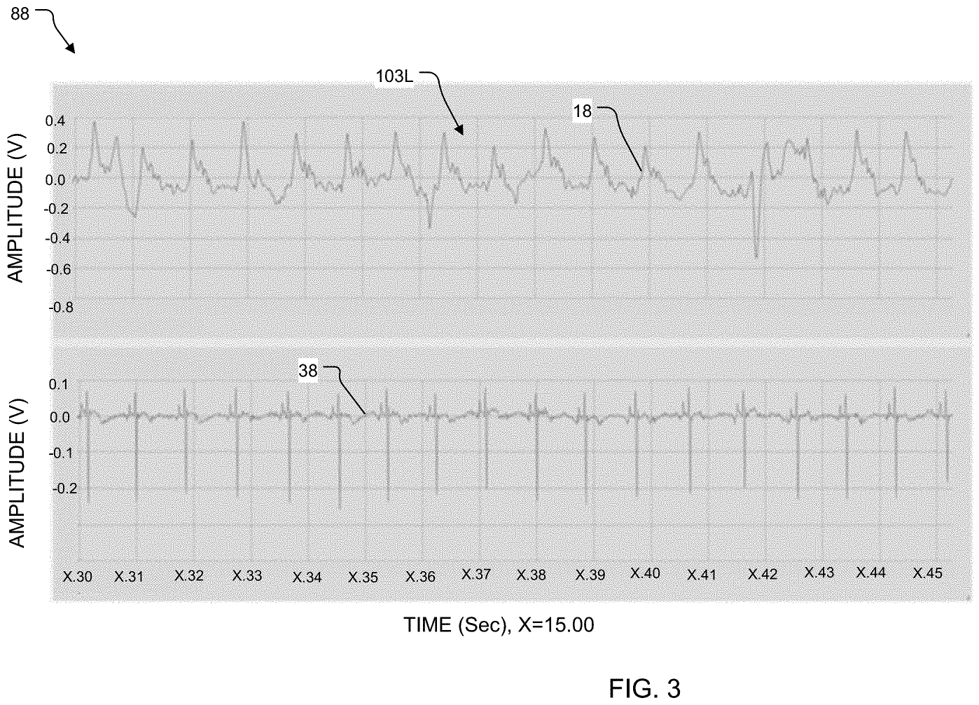

FIG. 3 shows plots of data within an exemplary DUT dataset 88 collected over a period of 15 seconds. At the top is a plot of earbud signals 18 obtained by and sent from an earbud system 34 DUT, and at the bottom is a plot of a reference signal (here, ECG signals 38 obtained by and sent from an ECG system 24 reference clinical device). In this example, the clinical data system 10 has configured the clinical data recorder 50 to use the ECG signals 38 as a reference signal to synchronize the earbud signals 18 upon receiving the earbud signals 18 from the earbuds 103.

While this invention has been particularly shown and described with references to preferred embodiments thereof, it will be understood by those skilled in the art that various changes in form and details may be made therein without departing from the scope of the invention encompassed by the appended claims.

What is claimed is:

1. A synchronous clinical data collection, analysis, and reporting system (“clinical data system”), the clinical data system comprising:
a clinical data recorder that collects and records signal data of a patient obtained by and sent from reference clinical devices during a medical session, synchronizes the signal data from each of the reference clinical devices using a reference signal, and creates clinical datasets that include the signal data from each of the reference clinical devices and the reference signal;
one or more devices under test (DUT) that each obtain signal data from the patient during the session, wherein each DUT synchronizes its signal data using the reference signal and creates DUT datasets that each include the signal data from each DUT and the reference signal; and
a computing unit that receives the clinical datasets from the clinical data recorder and the DUT datasets sent from each of the DUTs, computes a normalized reference signal for each DUT dataset, and synchronizes the signal data of each DUT dataset with the signal data of the clinical datasets using the normalized reference signal computed for each DUT dataset.

2. The clinical data system of claim 1, wherein the clinical data recorder is configured to obtain the reference signal from the signal data of one of the reference clinical devices.

3. The clinical data system of claim 2, wherein the reference signal from the signal data of one of the reference clinical devices is/are ECG signals of an ECG system reference clinical device.

4. The clinical data system of claim 1, wherein the clinical data recorder is configured to use the reference signal from the signal data of one of the DUTs.

5. The clinical data system of claim 4, wherein the reference signal from the signal data of one of the DUTs is/are earbud signals detected by and sent from an earbud system DUT.

6. The clinical data system of claim 1, wherein the computing unit records the DUT datasets and provides an interface that enables operators of the DUTs to access anonymized versions of the DUT datasets in real time during the session and after all DUT datasets have been received.

7. The clinical data system of claim 1, wherein the computing unit provides an interface that enables an administrator of the system to access anonymized versions of the clinical datasets in real time during the session and after all clinical datasets have been received.

8. The clinical data system of claim 1, wherein the computing unit computes a normalized reference signal for each DUT dataset by identifying DUT datasets and one or more clinical datasets obtained over substantially a same time period, and calculating and subtracting delays between the reference signal of the clinical datasets and the reference signal of each DUT dataset obtained over substantially the same time period, the result of which is/are the normalized reference signal for each DUT dataset.

9. The clinical data system of claim 1, wherein the computing unit calibrates the signal data of each DUT dataset upon determining that delays between the signal data and reference signal of each DUT dataset collected over a same time period are above a threshold value.

10. The clinical data system of claim 1, wherein when the reference signal in each DUT dataset is an ECG signal from an ECG system, the computing unit either calibrates each DUT dataset upon determining that delays between the signal data and reference signal of each DUT dataset collected over a same time period are above a threshold value, or upon determining that delays between the signal data and the reference signal of each DUT dataset collected over the same time period change more than a threshold amount over a range of successive heartbeats in the ECG signal as the reference signal.

11. The clinical data system of claim 10, wherein the computing unit calibrates each DUT dataset by either identifying a signal feature in or introducing a signal feature into both the signal data and the reference signal of each DUT dataset collected over the same time period, and then identifying a difference in time when the signal feature occurs in the signal data and in the reference signal.

12. The clinical data system of claim 1, wherein the computing unit calibrates each clinical dataset upon determining that delays between the signal data and reference signal of each clinical dataset are above a threshold value.

13. The clinical data system of claim 1, wherein when the reference signal in each clinical dataset is an ECG signal from an ECG system, the computing unit either calibrates the clinical dataset upon determining that delays between the signal data and reference signal of each clinical dataset are above a threshold value, or upon determining that delays between the signal data and the reference signal of each clinical dataset change more than a threshold amount over a range of successive heartbeats in the ECG signal as the reference signal.

14. The clinical data system of claim 13, wherein the computing unit calibrates each clinical dataset by either identifying a signal feature in or introducing a signal feature into both the signal data and the reference signal of each clinical dataset, and then identifying a difference in time when the signal feature occurs in the signal data and in the reference signal of each clinical dataset.

15. A synchronous clinical data collection, analysis, and reporting method, the method comprising:
collecting signal data of a patient obtained by and sent from reference clinical devices during a medical session, synchronizing the signal data from each of the reference clinical devices using a reference signal, and creating clinical datasets that include the signal data from each of the reference clinical devices and the reference signal;
collecting signal data of the patient obtained by and sent from one or more devices under test (DUT) during the session, synchronizing the signal data from each DUT using the same reference signal, and creating DUT datasets for each DUT that include the signal data from each DUT and the reference signal;
computing a normalized reference signal for each DUT dataset, based on differences in time between the reference signals of each DUT dataset collected over a same time period and the reference signals of clinical datasets collected over the same time period as each DUT dataset; and
synchronizing the signal data of each DUT dataset with the signal data of the clinical datasets using the normalized reference signal computed for each DUT dataset.

16. The method of claim 15, further comprising obtaining the reference signal from the signal data of one of the reference clinical devices.

17. The method of claim 15, further comprising obtaining the reference signal from the signal data of one of the DUTs.

18. The method of claim 17, wherein obtaining the reference signal from the signal data of one of the DUTs comprises obtaining the reference signal from earbud signals detected by and sent from an earbud system DUT.

19. The method of claim 15, further comprising recording the DUT datasets and providing an interface that enables operators of the DUTs to access anonymized versions of the DUT datasets in real time and after all DUT datasets have been recorded.

20. The method of claim 15, further comprising recording the clinical datasets and providing an interface that enables an administrator to access anonymized versions of the clinical datasets in real time and after all clinical datasets have been recorded.
